(19) United States
(12) Patent Application Publication
(10) Pub. No.: US 2012/0321582 A1
(43) Pub. Date: Dec. 20, 2012

(54) AIR SANITIZER AND MALODOUR REMOVAL FORMULATION

(75) Inventor: Wah Boon Low, Singapore (SG)

(73) Assignee: ANTIBAC LABORATORIES PTE LTD, Singapore (SG)

(21) Appl. No.: 13/580,617

(22) PCT Filed: May 25, 2010
(86) PCT No.: PCT/SG2010/000195

§ 371 Date: Aug. 22, 2012

(30) Foreign Application Priority Data
Mar. 5, 2010 (SG) 201001592-3

(51) Int. Cl. A61L 9/014

(52) U.S. Cl. 424/76.8

(57) ABSTRACT

There is provided a formulation for sanitizing air and reducing malodour in the air. The formulation comprises a malodour absorbing compound, an antimicrobial compound, wherein the antimicrobial compound is chemically incompatible with the malodour absorbing compound when mixed alone together, a solubiliser, and a chelating agent for the malodour absorbing compound, wherein the chelating agent prevents the formation of precipitate or a cloudy solution when the malodour absorbing compound is mixed with the antimicrobial compound in the presence of the solubiliser.

### FIELD OF INVENTION

The invention relates to a formulation for sanitizing air and reducing malodour in the air.

### BACKGROUND

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

Common indoor sources of malodours include tobacco smoke (amines e.g. nicotine), pungent foods such as garlic and onions (thio-compounds e.g. allicin) and stale fish, lavatories and human sweat (acids e.g. isovaleric acid). Airborne and surface malodours, when perceived by a person, generally result in detrimental effects to the mental and/or physical well-being of that person, particularly when the malodours exist indoor where air circulation is insufficient to vent the malodours to an outdoor environment. With people spending most of their time indoors, this poses a greater health risk especially for people who are the young, elderly and chronically ill suffering from asthmatic, respiratory or cardiovascular diseases.

Methods and compositions for suppressing, eliminating, or counteracting airborne malodours are well-known in the art. While a number of odour elimination methods have been developed, the most popular odour elimination method is by contacting the malodourants with a deodourising composition comprising one or more malodour counteractant.

A wide variety of deodourising compositions are known in the art. Odour masking is the intentional concealment of one odour by the addition of another. The masking of odours is typically accomplished by using perfumes or fragrances. However, large quantities of fragrances are needed to ensure that the malodours are no longer noticeable, or are suitably masked. Further, these masking techniques do nothing to remove or modify the source of the malodours.

Therefore, it is desirable to provide a formulation for sanitizing air and reducing malodour in the air.

### SUMMARY OF THE INVENTION

Throughout this document, unless otherwise indicated to the contrary, the terms “comprising”, “consisting of”, and the like, are to be construed as non-exhaustive, or in other words, as meaning “including, but not limited to”.

In a first aspect of the present invention, there is provided a formulation for sanitizing air and reducing malodour in the air. The formulation comprises a malodour absorbing compound, an antimicrobial compound, wherein the antimicrobial compound is chemically incompatible with the malodour absorbing compound when mixed alone together, a solubiliser, and a chelating agent for the malodour absorbing compound, wherein the chelating agent prevents the formation of precipitate or a cloudy solution when the malodour absorbing compound is mixed with the antimicrobial compound in the presence of the solubiliser.

In a second aspect of the present invention, there is provided a method forming a formulation for sanitizing air and reducing malodour in the air. The method comprises providing a solution of malodour absorbing compound, adding a solution of a chelating agent to the solution of malodour absorbing compound, adding a solution of solubiliser, and adding a solution of an antimicrobial compound to the solution containing the malodour absorbing compound, the chelating agent and the solubiliser, wherein the antimicrobial compound is chemically incompatible with the malodour absorbing compound when mixed alone together in the absence of the solution of chelating agent, and further wherein the solution of the chelating agent prevents the formation of precipitate or a cloudy solution when the solution of the malodour absorbing compound is mixed with the solution of the antimicrobial compound in the presence of the solubiliser.

### DETAILED DESCRIPTION

The invention relates to a formulation for sanitizing air and reducing malodour in the air.

Briefly, malodour in the air may be reduced by absorbing and removing the malodour from the air.

Sanitizing the air refers to the action of killing airborne bacteria or microorganisms which are often a source of odours. It is to be understood and appreciated that a total kill (i.e. 100%) of airborne bacteria is, in practice, almost never achieved. In the context of the present invention, the air is said to be sanitized when at least 85% kill of airborne bacteria has been achieved for a given period of time.

Therefore, if the quantity of malodours present in the air can be reduced and at the same time, the quantity of odour-causing bacteria can also be reduced, the quality of air can be greatly enhanced. It is with these objectives that the present invention has been developed.

In accordance with a first embodiment of the invention, there is provided a formulation for sanitizing air and reducing malodour in the air. The formulation comprises a malodour absorbing compound, an antimicrobial compound, wherein the antimicrobial compound is chemically incompatible with the malodour absorbing compound when mixed alone together, a solubiliser, and a chelating agent for the malodour absorbing compound, wherein the chelating agent prevents the formation of precipitate or a cloudy solution when the malodour absorbing compound is mixed with the antimicrobial compound in the presence of the solubiliser.

The malodour absorbing compound is a highly effective odour absorber based on the zinc salt of ricinoleic acid activated by an amino acid. Advantageously, the malodour absorbing compound is water soluble. Preferably, the malodour absorbing compound is zinc ricinoleate (trade name TEGO® Sorb A30 by Evonik Goldschmidt Corporation) and is present in less than 5 wt % based on the total weight of the formulation. Zinc ricinoleate controls osmogenes like hydrogen sulphide, mercaptan, thioether, isovaleric acid and ammonia effectively by a chemical reaction and odour emissions into the environment are prevented. Zinc ricinoleate has no bacterial or fungicidal properties.

Advantageously, the microbiocidal spectrum of the antimicrobial compound covers Gram-positive and Gram-negative bacteria, yeast and fungi. Preferably, the antimicrobial compound combats viruses such as HBV/HIV, Vaccinia, Herpes simplex, BVDV (Bovine Viral Diarrhoea Virus)/HCV, Rota Virus and Bovine Corona Virus/SARS-CoV. Advantageously, the antimicrobial compound is water soluble. In one embodiment, the antimicrobial compound includes amines, N—C₁₀₋₁₆-alkyltrimethylenedi, -reaction products with chloro-acetic acid. More preferably, the antimicrobial compound is N-alkyl aminopropyl glycine (trade name REWOCID® WK30 by Evonik Goldschmidt Corporation) and is present in less than 10 wt % of the total weight of the formulation.

When the malodour absorbing compound zinc ricinoleate is mixed alone with the antimicrobial compound N-alkyl aminopropyl glycine, precipitates start to form and a cloudy solution is obtained. This cloudy solution is unstable and the formation of precipitates demonstrates that a chemical reaction between zinc ricinoleate and N-alkyl aminopropyl glycine has taken place. As a consequence of the chemical reaction, zinc ricinoleate may not absorb malodours as effectively as before. Similarly, N-alkyl aminopropyl glycine may not function effectively as an antimicrobial agent compared to prior to mixing.

To promote the stability of zinc ricinoleate in N-alkyl aminopropyl glycine, a chelating agent for zinc ricinoleate is first mixed with zinc ricinoleate before mixing the chelated zinc ricinoleate solution with N-alkyl aminopropyl glycine. However, it has been found that a cloudy solution is obtained nevertheless.

However, when a solubiliser and water are subsequently added to the solution containing the chelated zinc ricinoleate and N-alkyl aminopropyl glycine, a clear solution is now obtained. The chelating agent prevents the formation of precipitate or a cloudy solution when zinc ricinoleate is mixed with N-alkyl aminopropyl glycine in the presence of the solubiliser. Zinc ricinoleate is now made miscible in N-alkyl aminopropyl glycine with the addition of the chelating agent in the presence of the solubiliser, producing a clear solution. The chelating agent does not impair the malodour absorbing ability of zinc ricinoleate. Advantageously, the chelating agent is biodegradable and stable over a wide range of pH. Preferably, the chelating agent is sodium iminodisuccinate (trade name Baypure® CX from Bayer Corporation) and is present in less than 5 wt % of the total weight of the formulation.

Preferably, the solubiliser is primarily a combination of a vegetable-derived hydrophilic emollient ester and polyethylene glycol (PEG) derivatives of hydrogenated castor oil. More preferably, the solubiliser is an ethoxylated palm kernel oil. More preferably, the solubiliser is a combination of PEG-45 palm kernel glycerides and PEG-6 capric/caprylic glycerides (trade name Cromollient SG90 by Croda Inc) and is present in less than 40 wt % of the total weight of the formulation.

The solubility of the formulation may further be enhanced with the addition of an alcohol and/or glycol. Preferably, the formulation further comprises less than 5 wt % ethanol and 1 wt % dipropylene glycol based on the total weight of the formulation.

Optionally, the formulation further comprises fragrance, which is oil-based. The fragrance not only introduces a pleasant smell in the air but also helps to invigorate senses. Such fragrances generally encourage relaxation, soothe mind and body, and enhance overall state of well-being. Preferably, the fragrance is present in less than 30 wt % based on the total weight of the formulation.

Advantageously, the solubiliser also aids in the solubility of the oil-based fragrance in the water-based formulation.

Optionally, preservatives may be added to preserve the shell-life of the formulation. In one embodiment, the preservative is dimethylol dimethylhydantoin or DMDM Hydantoin for short (trade name Glydant® by Lonza Group Ltd) and is present in less than 0.5 wt % based on the total weight of the formulation.

Advantageously, a defoamer is added to the water-based formulation to provide effective foam control. The defoamer acts to reduce air entrainment during manufacture, handling and application of the formulation. Preferably, the defoamer is polymeric silicone (trade name Dow Corning® 74 Additive) and is present in less than 1 wt % based on the total weight of the formulation.

### EXAMPLES

Comparative examples of the formulations are illustrated below. In all examples, the ingredients (represented by their respective trade names) are first mixed together and stirred until a uniform mixture is obtained. Deionised water is then slowly added to the mixture and mixed until a homogeneous solution is obtained. Examples No. 4 and 8 are provided according to the present invention.

### Example 1

| Trade Name | Chemical Name | Function | Wt % |
|---|---|---|---|
| Japanese | Fragrance | Aroma fragrance | 15.0 |
| Green Tea | | | |
| Cromollient | PEG-45 Palm Kernel | Solubiliser | 28.0 |
| SG 90 | Glycerides & PEG-6 | | |
| | Capric/Caprylic | | |
| | Glycerides | | |
| Glydant ® | DMDM Hydantoin | Preservative | 0.30 |
| Water, DI | — | Solvent | Bal |

### Example 2

| Trade Name | Chemical Name | Function | Wt % |
|---|---|---|---|
| TEGO ® | Zinc Ricinoleate | Malodour Absorber | 2.0 |
| Sorb A30 | | | |
| Japanese | Fragrance | Aroma fragrance | 15.0 |
| Green Tea | | | |
| Cromollient | PEG-45 Palm Kernel | Solubiliser | 28.0 |
| SG 90 | Glycerides & PEG-6 | | |
| | Capric/Caprylic | | |
| | Glycerides | | |
| Glydant ® | DMDM Hydantoin | Preservative | 0.30 |
| Water, DI | — | Solvent | Bal |

### Example 3

| Trade Name | Chemical Name | Function | Wt % |
|---|---|---|---|
| Rewocid ® | N-Alkyl aminopropyl | Antimicrobial | 7.50 |
| WK30 | glycine | compound | |
| Japanese | Fragrance | Aroma fragrance | 15.0 |
| Green Tea | | | |
| Cromollient | PEG-45 Palm Kernel | Solubiliser | 28.0 |
| SG 90 | Glycerides & PEG-6 | | |
| | Capric/Caprylic | | |
| | Glycerides | | |
| Glydant ® | DMDM Hydantoin | Preservative | 0.30 |
| Water, DI | — | Solvent | Bal |

### Example 4

| Trade Name | Chemical Name | Function | Wt % |
|---|---|---|---|
| Rewocid ® | N-Alkyl aminopropyl | Antimicrobial | 7.50 |
| WK30 | glycine | compound | |
| TEGO ® | Zinc Ricinoleate | Malodour Absorber | 2.0 |
| Sorb A30 | | | |
| Baypure ® | Sodium | Chelating Agent | 3.0 |
| CX | Iminodisuccinate | | |
| Japanese | Fragrance | Aroma fragrance | 15.0 |
| Green Tea | | | |
| Cromollient | PEG-45 Palm Kernel | Solubiliser | 28.0 |
| SG 90 | Glycerides & PEG-6 | | |
| | Capric/Caprylic | | |
| | Glycerides | | |
| Glydant ® | DMDM Hydantoin | Preservative | 0.30 |
| Water, DI | — | Solvent | Bal |

### Example 5

| Trade Name | Chemical Name | Function | Wt % |
|---|---|---|---|
| Lady Apple | Fragrance | Aroma fragrance | 15.0 |
| Apple Blossom | Fragrance | Aroma fragrance | 5.0 |
| Cromollient | PEG-45 Palm Kernel | Solubiliser | 36.0 |
| SG 90 | Glycerides & PEG-6 | | |
| | Capric/Caprylic | | |
| | Glycerides | | |
| Glydant ® | DMDM Hydantoin | Preservative | 0.30 |
| Water, DI | — | Solvent | Bal |

### Example 6

| Trade Name | Chemical Name | Function | Wt % |
|---|---|---|---|
| TEGO ® | Zinc Ricinoleate | Malodour Absorber | 2.0 |
| Sorb A30 | | | |
| Lady Apple | Fragrance | Aroma fragrance | 15.0 |
| Apple Blossom | Fragrance | Aroma fragrance | 5.0 |
| Cromollient | PEG-45 Palm Kernel | Solubiliser | 36.0 |
| SG 90 | Glycerides & PEG-6 | | |
| | Capric/Caprylic | | |
| | Glycerides | | |
| Glydant ® | DMDM Hydantoin | Preservative | 0.30 |
| Water, DI | — | Solvent | Bal |

### Example 7

| Trade Name | Chemical Name | Function | Wt % |
|---|---|---|---|
| Rewocid ® | N-Alkyl aminopropyl | Antimicrobial | 7.50 |
| WK30 | glycine | compound | |
| Lady Apple | Fragrance | Aroma fragrance | 15.0 |
| Apple Blossom | Fragrance | Aroma fragrance | 5.0 |
| Cromollient | PEG-45 Palm Kernel | Solubiliser | 36.0 |
| SG 90 | Glycerides & PEG-6 | | |
| | Capric/Caprylic | | |
| | Glycerides | | |
| Glydant ® | DMDM Hydantoin | Preservative | 0.30 |
| Water, DI | — | Solvent | Bal |

### Example 8

| Trade Name | Chemical Name | Function | Wt % |
|---|---|---|---|
| Rewocid ® | N-Alkyl aminopropyl | Antimicrobial | 7.50 |
| WK30 | glycine | compound | |
| TEGO ® | Zinc Ricinoleate | Malodour Absorber | 2.0 |
| Sorb A30 | | | |
| Baypure ® | Sodium | Chelating Agent | 3.0 |
| CX | Iminodisuccinate | | |
| Lady Apple | Fragrance | Aroma fragrance | 15.0 |
| Apple Blossom | Fragrance | Aroma fragrance | 5.0 |
| Cromollient | PEG-45 Palm Kernel | Solubiliser | 36.0 |
| SG 90 | Glycerides & PEG-6 | | |
| | Capric/Caprylic | | |
| | Glycerides | | |
| Glydant ® | DMDM Hydantoin | Preservative | 0.30 |
| Water, DI | — | Solvent | Bal |

### Results

In the exemplary formulations illustrated above, tests in Sniff Chamber (Square Acrylic Chamber 40 cm×40 cm×40 cm) were conducted. The tests were conducted to evaluate the effect of each test formulation in removing/reducing ammonia, a common malodour in the air.

Five millilitres (ml) of each test formulation was put in a commercially available air purifier container, and in this case, the air purifier container being MagicBall (Antibac2K®) and 495 ml of deionised water was added (1:100 dilution). The MagicBall and 1 ml ammonia, 25% solution (test substance containing the malodour) were then put inside the Acrylic Chamber. The smell was evaluated by 5 trained evaluators.

Each evaluator was asked to rate the intensity of the malodour after 1 second, 1 minute and 10 minutes, respectively.

The evaluation rating is used as follows:—

5: overpowering
4: strong
3: moderate
2: weak
1: not detectable

Following were the results of the Sniff Test (mean value of 5 judgements):

Test Substance: Ammonia, 1 ml of 25% Solution in Sniff Chamber

| Example | After 10 seconds | After 1 minute | After 10 minutes |
|---|---|---|---|
| 1 | 5 | 4.4 | 4.2 |
| 2 | 5 | 3.6 | 2.6 |
| 3 | 5 | 3.6 | 3.2 |
| 4 | 5 | 2.0 | 1.6 |
| 5 | 5 | 4.0 | 3.8 |
| 6 | 5 | 3.4 | 3.0 |
| 7 | 5 | 3.6 | 3.2 |
| 8 | 5 | 1.8 | 1.2 |

Based on above results obtained, it is clear that Example no. 4 and 8 (i.e. formulations according to the present invention) showed that greatest reduction in the intensity of the malodour after a specified period of time.

To further demonstrate the antimicrobial efficacy of the formulations according to the present invention, various microorganisms were tested against the formulations in laboratory. The results are presented below.

### Example 9

The following formulation of Antibac2K® Fruity Apple using N-alkyl aminopropyl glycine (100 times dilution) was tested:

| Trade Name | Chemical Name | Function | Wt % |
|---|---|---|---|
| Lady Apple and | Fragrance | Aroma fragrance | 25.0 |
| Apple Blossom | | | |
| — | Ethanol | Solubility enhancer | 3.0 |
| — | Dipropylene Glycol | Solubility enhancer | 0.50 |
| Cromollient | PEG-45 Palm Kernel | Solubilizer | 34.0 |
| SG 90 | Glycerides & PEG-6 | | |
| | Capric/Caprylic | | |
| | Glycerides | | |
| DC 74 | Silicone defoamer | Defoamer | 0.50 |
| Baypure ® | Sodium | Chelating Agent | 3.0 |
| CX 100/34 | Iminodisuccinate | | |
| Rewocid ® | N-Alkyl aminopropyl | Antimicrobial | 7.50 |
| WK30 | glycine | compound | |
| TEGO ® | Zinc Ricinoleate | Malodour Absorber | 2.0 |
| Sorb A30 | | | |
| Glydant ® | DMDM Hydantoin | Preservative | 0.30 |
| Water, DI | — | Solvent | Bal |

Test microorganism: Escherichia coli

| | Count of Surviving Test | Percentage Kill of text |
|---|---|---|
| Contact Time (min) | Microorganisms (CFU/ml) | Microorganisms (%) |
| 0 | 6.3 × 10⁵ | 0 |
| 1 | 0 | 99.99 |
| 5 | 0 | 99.99 |

Test microorganism: Staphylococcus aureus

| | Count of Surviving Test | Percentage Kill of text |
|---|---|---|
| Contact Time (min) | Microorganisms (CFU/ml) | Microorganisms (%) |
| 0 | 2.5 × 10⁵ | 0 |
| 1 | 0 | 99.99 |
| 5 | 0 | 99.99 |

Test microorganism: Bacillus subtilis

| | Count of Surviving Test | Percentage Kill of text |
|---|---|---|
| Contact Time (min) | Microorganisms (CFU/ml) | Microorganisms (%) |
| 0 | 3.8 × 10⁴ | 0 |
| 1 | 0 | 99.99 |
| 5 | 0 | 99.99 |

Test microorganism: Salmonella enteritidis

| | Count of Surviving Test | Percentage Kill of text |
|---|---|---|
| Contact Time (min) | Microorganisms (CFU/ml) | Microorganisms (%) |
| 0 | 2.1 × 10⁵ | 0 |
| 1 | 0 | 99.99 |
| 5 | 0 | 99.99 |

### Example 10

The following is formulation of Antibac2K® Fruity Apple using N-alkyl aminopropyl glycine (500 times dilution) was tested:

| Trade Name | Chemical Name | Function | Wt % |
|---|---|---|---|
| Lady Apple and | Fragrance | Aroma fragrance | 25.0 |
| Apple Blossom | | | |
| — | Ethanol | Solubility enhancer | 3.0 |
| — | Dipropylene Glycol | Solubility enhancer | 0.50 |
| Cromollient | PEG-45 Palm Kernel | Solubilizer | 34.0 |
| SG 90 | Glycerides & PEG-6 | | |
| | Capric/Caprylic | | |
| | Glycerides | | |
| Dow Corning ® | Silicone defoamer | Defoamer | 0.50 |
| 74 Additive | | | |
| Baypure ® | Sodium | Chelating Agent | 3.0 |
| CX 100/34 | Iminodisuccinate | | |
| Rewocid ® | N-Alkyl aminopropyl | Antimicrobial | 7.50 |
| WK30 | glycine | compound | |
| TEGO ® | Zinc Ricinoleate | Malodour Absorber | 2.0 |
| Sorb A30 | | | |
| Glydant ® | DMDM Hydantoin | Preservative | 0.30 |
| Water, DI | — | Solvent | Bal |

Test microorganism: Escherichia coli

| | Count of Surviving Test | Percentage Kill of text |
|---|---|---|
| Contact Time (min) | Microorganisms (CFU/ml) | Microorganisms (%) |
| 0 | 6.3 × 10⁵ | 0 |
| 1 | 0 | 99.99 |
| 5 | 0 | 99.99 |

Test microorganism: Staphylococcus aureus

| | Count of Surviving Test | Percentage Kill of text |
|---|---|---|
| Contact Time (min) | Microorganisms (CFU/ml) | Microorganisms (%) |
| 0 | 2.5 × 10⁵ | 0 |
| 1 | 0 | 99.99 |
| 5 | 0 | 99.99 |

Test microorganism: Bacillus subtilis

| | Count of Surviving Test | Percentage Kill of text |
|---|---|---|
| Contact Time (min) | Microorganisms (CFU/ml) | Microorganisms (%) |
| 0 | 3.8 × 10⁴ | 0 |
| 1 | 0 | 99.99 |
| 5 | 0 | 99.99 |

Test microorganism: Salmonella enteritidis

| | Count of Surviving Test | Percentage Kill of text |
|---|---|---|
| Contact Time (min) | Microorganisms (CFU/ml) | Microorganisms (%) |
| 0 | 2.1 × 10⁵ | 0 |
| 1 | 0 | 99.99 |
| 5 | 0 | 99.99 |

The results of the afore-described formulation clearly provide that air quality is greatly enhanced with the synergy of the malodour absorbing compound and the antimicrobial compound according to the present invention, compared to the effects of the individual compounds alone.

The present formulation is a water-based antimicrobial and malodour removal formulation with botanical extracts. It is non-toxic and non-carcinogenic, non-irritant, non-corrosive, biodegradable, easily handled and non-tainting. As such, the formulation is not harmful to human, animals and the environment. This powerful formulation has a broad spectrum of antimicrobial efficacy, where it eliminates Gram-positive and Gram-negative bacteria, yeast, fungi and enveloped viruses.

Although the foregoing invention has been described in some detail by way of illustration and example, and with regard to one or more embodiments, for the purposes of clarity of understanding, it is readily apparent to those of ordinary skill in the art in light of the teachings of this invention that certain changes, variations and modifications may be made thereto without departing from the spirit or scope of the invention as described in the appended claims.

1-26. (canceled)

27. A formulation for sanitizing air and reducing malodour in the air, the formulation comprising:
a malodour absorbing compound being a zinc salt of ricinoleic acid;
an antimicrobial compound, wherein the antimicrobial compound is chemically incompatible with the malodour absorbing compound when mixed alone together and is selected from the group consisting of amines, N—C₁₀₋₁₆-alkyltrimethylenedi, -reaction products with chloro-acetic acid;
a solubiliser; and
a chelating agent for the malodour absorbing compound, wherein the chelating agent prevents the formation of precipitate or a cloudy solution when the malodour absorbing compound is mixed with the antimicrobial compound in the presence of the solubiliser.

28. The formulation of claim 27, wherein the malodour absorbing compound is zinc ricinoleate.

29. The formulation of claim 28, wherein the formulation comprises less than 5 wt % (total weight percentage) of zinc ricinoleate.

30. The formulation of claim 27, wherein the antimicrobial compound is N-alkyl aminopropyl glycine.

31. The formulation of claim 30, wherein the formulation comprises less than 10 wt % (total weight percentage) of N-alkyl aminopropyl glycine.

32. The formulation of claim 27, wherein the chelating agent is sodium iminodisuccinate.

33. The formulation of claim 32, wherein the formulation comprises less than 5 wt % (total weight percentage) of sodium iminodisuccinate.

34. The formulation of claim 27, wherein the solubiliser is selected from the group consisting of a combination of a vegetable-derived hydrophilic emollient ester and polyethylene glycol (PEG) derivatives of hydrogenated castor oil, alcohol, glycol, and mixture therein.

35. The formulation of claim 34, wherein the solubiliser is an ethoxylated palm kernel oil.

36. The formulation of claim 35, wherein the solubiliser is a combination of PEG-45 palm kernel glycerides and PEG-6 capric/caprylic glycerides.

37. The formulation of claim 36, wherein the formulation comprises less than 40 wt % (total weight percentage) of PEG-45 palm kernel glycerides and PEG-6 capric/caprylic glycerides.

38. The formulation of claim 37, wherein the solubiliser further comprises ethanol.

39. The formulation of claim 38, wherein the formulation comprises less than 5 wt % (total weight percentage) ethanol.

40. The formulation of claim 39, wherein the solubiliser further comprises dipropylene glycol.

41. The formulation of claim 40, wherein the formulation comprises less than 1 wt % (total weight percentage) dipropylene glycol.

42. The formulation of claim 27, further comprising a fragrance.

43. The formulation of claim 42, wherein the formulation comprises less than 30 wt % (total weight percentage) of fragrance.

44. The formulation of claim 27, further comprising a preservative.

45. The formulation of claim 44, wherein the preservative is dimethylol dimethylhydantoin.

46. The formulation of claim 45, wherein the formulation comprises less than 0.5 wt % (total weight percentage) dimethylol dimethylhydantoin.

47. The formulation of claim 27, further comprising a defoamer.

48. The formulation of claim 47, wherein the defoamer is polymeric silicone.

49. The formulation of claim 48, wherein the formulation comprises less than 1 wt % (total weight percentage) polymeric silicone.

50. The formulation of claim 27, further comprising water.

51. A method of utilizing the formulation of claim 27, comprising:
providing the formulation as a solution for an air purifier.

52. A method for forming a formulation for sanitizing air and reducing malodour in the air, the method comprising:
providing a solution of malodour absorbing compound, the malodour absorbing compound being a zinc salt of ricinoleic acid;
adding a solution of a chelating agent to the solution of malodour absorbing compound;
adding a solution of solubiliser; and
adding a solution of an antimicrobial compound to the solution containing the malodour absorbing compound, solubiliser, and the chelating agent, wherein the antimicrobial compound is chemically incompatible with the malodour absorbing compound when mixed alone together in the absence of the solution of chelating agent and is selected from the group consisting of amines, N—C₁₀₋₁₆-alkyltrimethylenedi, -reaction products with chloro-acetic acid,
wherein the solution of the chelating agent prevents the formation of precipitate or a cloudy solution when the solution of the malodour absorbing compound is mixed with the solution of the antimicrobial compound in the presence of the solubiliser.
